(19) United States
(12) Patent Application Publication
(10) Pub. No.: US 2021/0104311 A1
(43) Pub. Date: Apr. 8, 2021

(54) PERSONALIZED ASSISTANCE FOR IMPAIRED SUBJECTS

(71) Applicant: KONINKLIJKE PHILIPS N.V., EINDHOVEN (NL)

(72) Inventors: Daniel Jason SCHULMAN, Jamaica Plain, MA (US); Saman PARVANEH, Danvers, MA (US); Mladen MILOSEVIC, Stoneham, MA (US); Portia E SINGH, Everett, MA (US); Ali Akbar Ahmad SAMADANI, Somerville, MA (US)

(21) Appl. No.: 16/772,237

(22) PCT Filed: Dec. 5, 2018
(86) PCT No.: PCT/EP2018/083599

§ 371 Date: Jun. 12, 2020

Related U.S. Application Data
(60) Provisional application No. 62/598,041, filed on Dec. 13, 2017.

(51) Int. Cl. G16H 20/70; A61B 5/11; A61B 5/16; A61B 5/00; G09B 19/00

(52) U.S. Cl. CPC G16H 20/70; A61B 5/1113; G09B 19/003; A61B 5/4088; A61B 5/162

(57) ABSTRACT

Techniques herein relate to personalized assistance for subjects with impairment(s). In various embodiments, a subject's state may be determined (402) from various signal(s). Based on the subject's state, a first computing device may be selected (404) from one or more computing devices available to the subject. Based on the subject's state and a policy associated with the subject, task(s) may be determined (406) that are performable by the subject with the aid of the first computing device. Task-selection input from the subject may be received (408) via the first computing device to initiate a triggered task. Task-engagement input may be received (412) via the first computing device from the subject that indicate completion of step(s) of the triggered task. The policy may be updated (414), e.g., using reinforcement learning, based on attribute(s) of the task-engagement inputs.

### TECHNICAL FIELD

Various embodiments described herein are directed generally to health care. More particularly, but not exclusively, various methods and apparatus disclosed herein relate to personalized assistance for subjects with impairment(s).

### BACKGROUND

Subjects living independently (e.g., living in their own home) and suffering from impairments such as cognitive impairment often have difficulty with the safe and successful performance of instrumental activities of daily living (“IADLs”) such as meal preparation, personal hygiene, and so forth. In-home technology can be used to provide automated reminders, guidance or other assistance, but determining the correct type, amount, and timing of assistance is difficult due to variability among individuals, their home environments, and context such as the availability of support, as well as changes in impairment over time.

For example, mild cognitive impairment (“MCI”) is defined as cognitive deficiency beyond the normal progression of aging, but not sufficient for a diagnosis of dementia. MCI is common, affecting about 18% of seniors in some estimates. Among individuals with MCI, trajectories of cognitive deficiency vary. While dementia is prevalent, and many individuals with MCI will eventually develop dementia, others may show a more gradual decline (similar to patterns of normal aging), and will never develop impairments severe enough for a diagnosis of dementia. The particular impairments of individuals with MCI also vary, and are broadly classified into amnestic, or memory-related impairments, and non-amnestic impairments. There is no broadly acceptable treatment to reduce impairments due to MCI, and it is typically managed as a chronic condition.

MCI has negative impact on health, wellness, and quality-of-life outcomes, especially on the ability of seniors to remain independent at home in daily life (“aging in-place”). One way in which MCI impacts these outcomes is by impairment in the ability of a subject to reliably perform IADLs. IADLs are tasks which build on the basic activities of daily living required for self-care, and allow a person to live independently in the community. Examples of IADLs include housework, preparing meals, managing finances, and self-management of chronic conditions including medication management. The specific set of IADLs which support an individual subject's independence can vary depending on characteristics of the individual (e.g. chronic health conditions requiring daily self-management), on the home environment, and on the individual's social context (e.g. the availability of support from an individual's social network, including emotional support and instrumental support for tasks such as shopping).

### SUMMARY

The present disclosure is directed to methods and apparatus for personalized assistance for subjects with impairment(s) such as cognitive impairment. In various embodiments, techniques described herein may be used to monitor and/or aid a subject that has been diagnosed as being at risk for, or suffering from, an impairment such as cognitive impairment. In various embodiments, one or more computing devices that are already operated by the subject and/or provided to the subject may be configured with software that, when executed, implements selected aspects of the present disclosure. These may include, for instance, laptop computers, tablet computers, mobile phones, desktop computers, set top boxes, “smart” televisions, standalone interactive speakers, and so forth.

When executed, the software may cause one or more of these computing devices to aid the subject in a variety of ways, such as assisting with performance of instrumental activities of daily living (“IADLs”). The software may cause these computing devices to provide output that includes prompts instructing the subject how to perform various IADL “tasks” that include one or more steps. For example, a series of prompts may be provided to instruct the subject how to cook a meal, conduct personal hygiene, get dressed, etc. IADL tasks may be set in the software by caregivers, clinicians, and/or the subject, e.g., tailored to the specific needs of the subject. Furthermore, IADL tasks might be selected from a generic list, or from a list tailored to potential needs of the subject, and be customized based on the needs of the subject. In some embodiments, steps of IADL tasks may also be customized, e.g., by caregivers, clinicians, and/or the subject. Steps of IADL could also be extracted from a generic list and may be customized. In some embodiments, audio and/or visual output provided at individual steps, at the outset of tasks, etc., may be customized, e.g., using pictures and/or videos of the subject's home in place of generic media, so that the subject is more familiar/comfortable with the guidance. In various embodiments, the subject may provide responsive input (also referred to as “task-engagement input”) that confirms performance of each step. Failure by the subject to provide task-engagement input, or at least timely task-engagement input, at each step may trigger a variety of different actions to be taken.

For example, in some embodiments, durations required for the subject to provide task-engagement input in response to prompts (e.g., indicating completion of a step of a task) and/or statistics computed based on those times may be evaluated to determine a measure of impairment of the subject. These ongoing measures of impairment may be monitored by clinicians, caregivers, etc. In some embodiments, one or more of these times and/or statistics may be applied as input across various types of machine learning classifiers, such as artificial neural networks, to classify the subject as having a particular level of impairment. If a subject's level of impairment changes, particularly if it appears the subject is deteriorating, caregivers and/or clinicians may be notified, e.g., via audio/visual output, and/or by email, text message, or other push notifications.

In addition, in some embodiments, the amount and type of guidance provided to a subject to perform IADL tasks may be selected based on attributes the subject, such as their level of impairment observed using techniques described herein. For subjects with mild (e.g., cognitive) impairment, a relatively small amount of guidance may be needed, e.g., in the form of relatively few prompts that must be responded to. On the other hand, subjects with more severe impairment may require more intense and/or granular instruction. Accordingly, techniques described herein facilitate selective provision of IADL guidance based on observations of subjects' levels of impairment. In particular, in some embodiments, a policy may be enacted that dictates the type and/or quantity of guidance that a subject receives. The policy may be used, for instance, to select a next action to take based on a subject's current state. Based on an estimate of subjects' ability to successfully perform tasks, e.g., detected based on attributes of responsive input provided by the subject, a measure of the subject's cognitive impairment (or any other type of impairment) may be determined. This measure may then be used to influence a policy associated with the subject, e.g., so that the policy evolves over time to suit the individual subject's condition.

A policy associated with a subject may be influenced by a variety of factors. In some embodiments, attributes of task-engagement inputs provided by the subject, such as the time required for the subject to respond to a prompt or an input modality employed by the subject to provide task-engagement inputs, may be considered. As an example, suppose over time a subject tends to take longer to complete steps of tasks, which in turn increases the time(s) required for the subject to provide task-engagement inputs. This may suggest some level of cognitive decline. Additionally or alternatively, in some embodiments, attributes of prompts provided to the subject, alone or in combination with aspect(s) of the subject's task-engagement inputs, may be considered. Attributes of the prompts may include measures of intrusiveness, output modalities, etc.

In some embodiments, a policy associated with a subject may be initially configured manually, e.g., by a clinician caring for the subject. Subsequently, the policy may be influenced (e.g., modified, computed, etc.) using various artificial intelligence algorithms and/or models. For example, in some embodiments, the policy may be influenced using one or more reinforcement learning techniques, which may attempt to choose a policy to optimize the expected cumulative value of some reward function. In some such embodiments, a reward function may be inspired by the System of Least Prompts, a strategy originally designed for teaching occupational skills to children with cognitive and developmental delays. This strategy is based on the idea that the least intrusive prompt that results in the desired response is desirable, and that prompts should be used in a graduated system, i.e. from least intrusive to most intrusive, until an appropriate response is received. A variety of different reinforcement learning techniques/algorithms may be applied. In some embodiments, a random forest batch-fitted Q learning algorithm may be employed. Such an algorithm may estimate a “Q function” of the policy, or an expected cumulative reward from taking a particular action while the subject is in a particular state, and following the specified policy thereafter. Additionally or alternatively, a policy may include one or more artificial neural networks that are configured to select an action based on a subject's state, and that are trained using reinforcement learning to select actions that optimize one or more reward values.

A subject's state may be indicative of a variety of different pieces of information related to the subject. In some embodiments, one or more presence sensors may be deployed throughout an environment such as a subject's home, such that an attribute of the subject's state may include the subject's last-known location. These presence sensors may take various forms, such as standalone presence sensors and/or presence sensors incorporated into other devices, such as computing devices operated by the subject, smart appliances (e.g., smart thermostats, smart refrigerators, etc.), and so forth. Other pieces of information that may or may not be included as part of a subject's state include but are not limited to a current date/time, a current task being performed (if one has been triggered) by the subject, the current step of a currently active task, recent detections of subject presence at the current location, outcomes of past tasks and/or task steps, attributes/statistics of times required for the subject to complete tasks and/or task steps, task outcome statistics, etc.

As noted above, a subject's state may be used, along with the policy, to select a next action (e.g., prompt the subject to perform a particular step of a current task, select a particular output modality for the prompt, etc.) to be taken by one or more computing devices configured with selected aspects of the present disclosure. For example, in some embodiments, a lookup table or other similar mechanisms may be used to select a next action. Additionally or alternatively, in some embodiments, one or more features of the subject's state may be applied as input across a trained machine learning model, such as an artificial neural network, to generate output. This output may include, for instance, probabilities associated with a plurality of potential responsive actions. In some embodiments the next action may be selected stochastically based on these probabilities.

Techniques described herein give rise to several technical advantages. For example, adjusting the type and/or volume of guidance (e.g., prompts, output modalities used to provide prompts, etc.) to suit a particular subject's condition may be more effective in guiding the subject through IADLs than simply providing the same amount of guidance across all subjects, regardless of their relative conditions. It also may conserve computing resources such as memory, processing cycles, network bandwidth, etc., by throttling the amount of guidance provided to a subject with relatively mild impairment. On the other hand, subjects' with increasing levels of impairment may be provided increasing amounts of guidance, as well as different types of guidance, to decrease negative outcomes, e.g., relating to performance of IADLs. In addition, by tracking a measure of a subject's impairment automatically and making this information available to clinicians, it is possible for medical personnel or a caregiver to more closely monitor a subject's progress and/or deterioration, and take remedial action if warranted. Some embodiments provide an additional technical advantage of reliability. In particular, as will be described in more detail below, in some embodiments, multiple computing devices operated by a subject may be used, some as “slave” devices and one or more as a “master” device. The master device may coordinate operation of the slave device(s) and may interact with cloud-based components. In the event of a failure of the master device or of an intermediate computer network, each of the slave devices may be configured with sufficient data and functionality to perform autonomously, so that a subject does not go without assistance due to technical difficulties (e.g., a Wi-Fi network failure).

Generally, in one aspect, a method may include the following operations: determining, from one or more signals, a state of a subject, wherein the subject is at risk for, or is suffering from, cognitive impairment; selecting, based on the state of the subject, a first computing device of one or more computing devices available to the subject; determining, based on the state of the subject and a policy associated with the subject, one or more tasks that are performable by the subject with the aid of the first computing device, wherein the policy is influenced by a measure of cognitive impairment exhibited by the subject; receiving, via the first computing device, task-selection input from the subject that initiates one or more of the tasks as a triggered task; receiving, via the first computing device, one or more task-engagement inputs from the subject that indicate completion of one or more steps of the triggered task; and updating the policy based on one or more attributes of the task-engagement inputs, wherein the updating includes applying a reinforcement learning technique to optimize a reward function.

In various embodiments, the method may further include providing, via one or more output components of the first computing device, one or more prompts to guide the subject through one or more of the steps of performing the triggered task. In various embodiments, the one or more prompts may be selected based at least in part on the policy associated with the subject, and updating the policy may further include updating the policy based at least in part on one or more attributes of the one or more prompts. In various embodiments, the one or more attributes of the one or more prompts may include a measure of intrusiveness.

In various embodiments, the one or more signals may include a signal from a presence sensor, and the state includes at least a last-detected location of the subject determined based on the signal from the presence sensor. In various embodiments, the first computing device may be further selected based on the policy associated with the subject. In various embodiments, the reinforcement learning technique comprises a random forest batch-fitted Q learning algorithm, although other algorithms, such as machine learning models, may be employed. For example, in various embodiments, the reinforcement learning technique may include a trained artificial neural network.

In various embodiments, the one or more attributes of the task-engagement inputs may include a reward or penalty determined based on a response time by the subject to provide a given task-engagement input of the task-engagement inputs. In various embodiments, the triggered task may include preparation of a meal. In various embodiments, the triggered task may include one or more of oral hygiene maintenance, medication ingestion, and adorning of clothing. In various embodiments, the triggered task may include pet care, appointment preparation, and household cleaning.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

In the drawings, like reference characters generally refer to the same parts throughout the different views. Also, the drawings are not necessarily to scale, emphasis instead generally being placed upon illustrating various principles of the embodiments described herein.

FIG. 1 schematically illustrates an example environment in which selected aspects of the present disclosure may be practiced, in accordance with various embodiments.

FIG. 2 depicts pseudocode that demonstrates one technique of reinforcement learning that may be used to update a policy associated with a subject, in accordance with various embodiments.

FIGS. 3A, 3B, 3C, and 3D depict example prompts that may be presented to a subject via a graphical user interface, in accordance with various embodiments.

FIG. 4 depicts an example method for practicing selected aspects of the present disclosure, in accordance with various embodiments.

FIG. 5 schematically illustrates an example computer system architecture on which selected aspects of the present disclosure may be implemented, in accordance with various embodiments.

### DETAILED DESCRIPTION

Subjects living independently (e.g., living in their own home) and suffering from impairments such as cognitive impairment often have difficulty with the safe and successful performance of instrumental activities of daily living (“IADLs”). In-home technology and/or wearable technology can be used to provide automated reminders, guidance or other assistance, but determining the correct type and amount of assistance is difficult due to variability among individuals and their home environments, as well as changes in impairment over time.

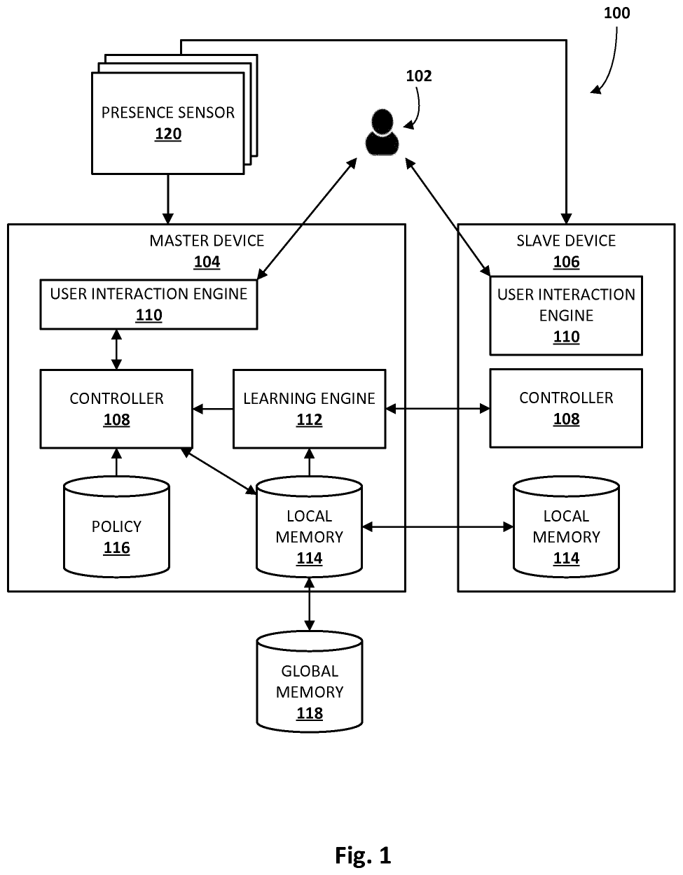

In view of the foregoing, various embodiments and implementations of the present disclosure are directed to personalized assistance for subjects with impairment(s) such as cognitive impairment. Referring to FIG. 1, a system 100 for providing assistance to a subject 102 at risk for, or already suffering from, one or more impairments (e.g., cognitive impairment) is depicted schematically. In various embodiments, system 100 may include one master devices 104 and one or more slave devices 106. In other embodiments, all devices may be treated the same. Master device(s) 104 and slave device(s) 106 may take various forms, including but not limited to tablet computers, desktop computers, laptop computers, smart phones, wearable devices (e.g., smart watches, smart glasses), set top boxes, smart televisions, standalone interactive speakers, and any other computing device that is capable of receiving input from a subject and providing output to the subject.

In various embodiments, master device 104 may include a controller 108, a user interaction engine 110, a learning engine 112, a local memory 114, and/or a policy 116. In other embodiments, one or more components 108-116 may be combined into fewer components, their respective functionalities may be split into additional components, and/or one or more components may be omitted. In various embodiments, master device 104 may be communicatively coupled, e.g., by way of one or more computing networks (not depicted), to a global memory 118, which may include memory of one or more remote computing systems forming part of a “cloud” computing system.

Controller 108 may be implemented using any combination of hardware or software. In some embodiments, controller 108 takes the form of one or more processors, such as one or more microprocessors, that are configured to execute software instructions in memory (not depicted) that cause controller 108 to perform selected aspects of the present disclosure. In other embodiments, controller 108 may take other forms, such as a software module, a field-programmable gate array (“FPGA”), an application-specific integrated circuit (“ASIC”), and so forth.

In various implementations, controller 108 may be configured to maintain a policy 116 associated with subject 102. Policy 116 may take various forms, such as a set of rules, one or more lookup tables mapping subject states to potential responsive actions, one or more machine learning models (e.g., artificial neural networks), and so forth. Controller 108 may also determine a current state of subject 102 based on a variety of signals (e.g., subject action, subject input, subject location, time, date, etc.), as well as update the subject's state, and may select actions to perform based on the subject's state and on policy 116.

User interaction engine 110 may be implemented with any combination of software and hardware, and may facilitate interaction between subject 102 and master device 104. For example, user interaction engine 110 may render one or more graphical user interfaces (“GUIs”) for presentation to subject 102 using one or more display devices (not depicted), as well as process inputs provided by subject 102 at those GUIs. And in various embodiments, user interaction engine 110 may provide output using other modalities than visual, such as audio (e.g., natural language output, audio prompts, etc.), haptic, etc. In some embodiments in which master device 104 (or slave device 106) takes the form of smart glasses or another similar device, user interaction engine 110 may provide augmented reality output, such as annotations that are presented visually to subject 102 overlaying the environment viewed by subject 102. User interaction engine 110 may receive input from subject 102 using a variety of modalities, such as via keyboard, mouse, touchscreen, audio input (e.g., spoken utterances), gestures (e.g., made with a smart phone), cameras (e.g., by making hand gestures), and so forth.

Also depicted as part of system 100 are one or more presence sensors 120. Presence sensors 120 may take various forms, including but not limited to passive infrared (“PIR”) sensors, weight- or pressure-based sensors (e.g., floor mats, chair mats, furniture covers and/or bedding that detect weight and/or pressure), cameras, light detectors, sensors that detect interaction with appliances (e.g., refrigerator door sensors, oven door sensors, regular door sensors, etc.), laser sensors, microphones, and so forth. Other types of presence sensors not specifically mentioned herein are contemplated.

Presence sensors 120 may be standalone sensors and/or incorporated with other devices. Various standalone presence sensors (or sensors integral with devices that are not full computing devices) may communicate with master device 104 and/or slave device 106 using various communication technologies, such as Wi-Fi, Bluetooth, ZigBee, Z-wave, etc. Additionally or alternatively, many computing devices (e.g., 104, 106) such as tablet computers, smart phones, smart glasses, standalone interactive speakers, laptop computers, etc., may include one or more built-in sensors that can effectively operate as presence sensors, such as cameras, microphones, etc. Additionally or alternatively, some appliances, such as smart thermostats or other detection devices, may include built-in presence sensors. In various embodiments, presence sensor(s) 120 may provide signals to master device 104 and/or slave device(s) 106 that indicate a detected presence, preferably of the subject-of-interest, at a particular location. As described herein, this detected subject location, along with a date and/or time associated with the detected location, may be included as part of a state of subject 102.

A subject's state may be a representation (e.g., a snapshot) that includes sufficient information to both select the next action to be performed and to facilitate adaptation of policy 116. In various embodiments, a subject's state may include a variety of different information, in addition to or instead of the subject's last-known location. For example, in some embodiments, a subject's state may include one or more of: the current time and date; the time and location of the last detected subject presence; a currently active task (if any), and step of the active task (if any). In some embodiments, each location data point in the subject's state may include a time of the subject's most recent presence detection. In some embodiments, for each task, a subject's state may include one or more of: the time and location of the most recent completed (successful or not) task; the outcome (e.g., success, failure, rejected) of the most recent task; and the proportion of each outcome (e.g., success, failure, rejected) among completed tasks in some time period (e.g., the last month). In some embodiments, for each step of a given task, the subject's state may include one or more of the following: the most recent duration to completion of the step (e.g., which may be the time elapsed between provision of a prompt instructing the subject how to perform the step and receipt of task-engagement input from the subject indicating completion of the step); the most recent outcome of the step (e.g., success, failure, rejected); statistics related to completion times over some time period (e.g., the last month), such as the mean and/or standard deviation; and the proportion of each outcome (e.g., success, failure, rejected) over the last month. Of course, these are simply examples of data points that may or may not be included in a subject's state. Other data points are contemplated.

In some embodiments, local memory 114 may be used to store a current and/or past states of subject 102. Additionally or alternatively, in some embodiments, local memory 114 may store “experiences” of subject 102, which may include a history of prior states, actions taken in response to the prior states, and in some cases, resulting states after those actions. As will be described below, in some embodiments, each “turn” of dialog between subject 102 and one or more of master device 104 and slave device 106 may be represented by a state/action/state triple, wherein the action (e.g., prompt) was taken in response to the state of subject 102 during that turn. Thus, in some embodiments, a series of state/action/state triples may be stored in local memory 114. This may be particularly important for reinforcement learning techniques described below, some of which may require at least temporary storage of past state/action/state triples in order to generate reward values and/or apply those reward values across multiple turns. In other embodiments, state/action pairs may be stored instead of state/action/state triples. In some embodiments, local memory 114 may receive state updates about subject 102 from controller 108 and/or from local memories 114 of slave device(s) 106.

As alluded to previously, in various embodiments, a subject's state may be used as input, e.g., by controller 108, to determine, e.g., based on policy 116, a next action to be taken by master device 104. Actions may include, for instance, providing various types of output to subject 102 to guide subject through performance of various IADLs. This output can include audio and/or visual prompts that offer subject 102 potential tasks (e.g., IADLs) based on the subject's current state, and/or provide steps of tasks that subject 102 should perform.

Suppose subject 102 walks into a kitchen at 8:00 AM. The subject's presence may be detected by one or more presence sensors 120 in the kitchen, such as by a refrigerator door being opened, by a PIR sensor mounted on a wall, by a camera integral with a tablet computer that is charging in the kitchen, etc. The subject's last known location (kitchen) and the current time (8:00 AM) may form part of the subject's current state. Based on this state, controller 108 may select a first computing device of one or more computing devices available to the subject. For example, if a tablet computer is charging in the kitchen and no other computing devices are determined to be closer to subject 102, the tablet computer may be selected. The tablet computer may be master device 104 or a slave device 106. The role of slave device(s) 106 will be described in more detail below.

Controller 108 next may determine, based on the state of the subject and policy 116, one or more tasks that are performable by subject 102 with the aid of the tablet computer. As noted above, in various embodiments, policy 116 is influenced by ongoing measures of cognitive impairment exhibited by subject 102. Accordingly, based on subject 102 suffering from some measure of cognitive impairment, policy 116 may dictate, e.g., to controller 108, that subject 102 should be provided with output, e.g., using the tablet computer, that suggests one or more tasks that subject 102 may wish to perform in his or her current state. At 8:00 AM in a kitchen, subject 102 may be presented with one or more breakfast options, such as making oatmeal, making pancakes, etc.

It may be the case the subject 102 desires a simpler breakfast such as fruit or cold cereal, in which case subject 102 may simply disregard (or affirmatively reject) the offered tasks. Or, in some cases, subject 102 may desire oatmeal but may feel confident in his or her ability to make it without guidance, and so may explicitly reject the offered task or provide some other indication that subject 102 doesn't need guidance. In some embodiments, e.g., where a measure of impairment of subject 102 satisfies some threshold (e.g., is sufficiently severe), or when the system is appropriately configured by a caregiver, one or more “smart” appliances (e.g., networked appliances such as stoves, ovens, microwaves, etc.) may be rendered inoperable unless subject 102 affirmatively selects an offered task that requires use of the smart appliances. If subject 102 attempts to cook something without receiving guidance, the smart appliances may prevent it.

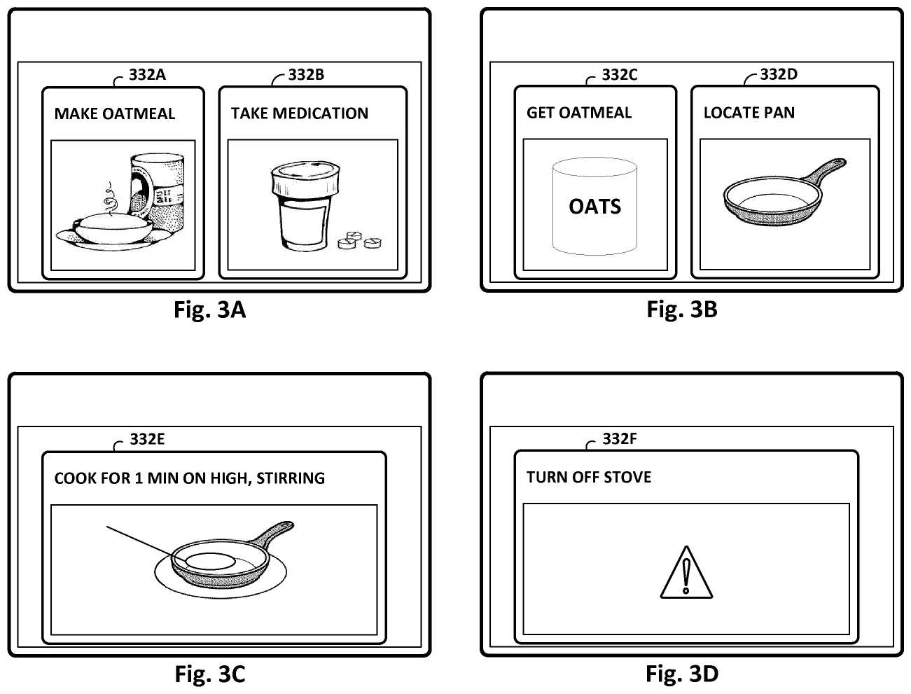

Suppose subject 102 selects an offered task, such as cooking some sort of breakfast, e.g., by tapping a graphical element on the tablet computer that corresponds to the task. Subject 102 may be presented with output (e.g., a series of prompts) to guide subject 102 through performance of the task. This output may be visual and/or audio. Examples of visual output that may be presented to guide subject 102 through preparation of oatmeal are depicted in FIGS. 3A-D. Audio output may be presented, for instance, as natural language output provided by a chatbot-like interface operating on the tablet computer (e.g., via a speaker on the tablet computer), or a nearby standalone interactive speaker, for instance.

As mentioned previously, providing a uniform amount of guidance to all subjects, regardless of their respective levels of impairment, may lead to less impaired subjects becoming frustrated with being “micromanaged,” while more severely impaired subjects may not receive sufficient guidance. Moreover, the needs of individual subjects may change as their levels of impairment change, e.g., deteriorate or even improve. Accordingly, in various embodiments, learning engine 112, which may be implemented using any combination of software and hardware, may be configured to apply various learning techniques to compute, re-compute, alter, tailor, customize, modify, or more generally, influence, policy 116 to suit the particular needs/impairment of subject 102.

Learning engine 112 may employ various different techniques to influence policy 116, depending on the nature of policy 116, preferences of subject 102, caregiver preferences, etc. In some embodiments, learning engine 112 is configured to compute policy 116 such that a reward function is optimized. In some embodiments, learning engine 112 may apply reinforcement learning to influence policy 116 such that subject 102 is provided with amounts of types of guidance that is tailored to a measure of impairment exhibited by subject 102.

For example, in some embodiments, learning engine 112 may employ a random forest batch-fitted Q learning algorithm. Such an algorithm may estimate a “Q function” of policy 116, or an expected cumulative reward from taking a particular action while subject 102 is in a particular state. In some such embodiments, a reward function may be inspired by the System of Least Prompts, a strategy originally designed for teaching occupational skills to children with cognitive and developmental delays. This strategy is based on the idea that the least intrusive prompt, that results in the desired response is desirable, and that prompts should be used in a graduated system, i.e. from least intrusive to most intrusive, until an appropriate response is received.

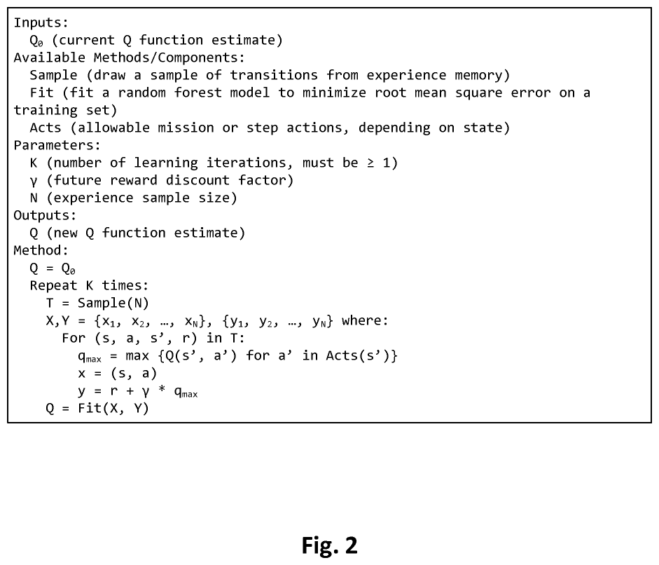

FIG. 2 depicts example pseudocode that demonstrates one example of how Q learning may be employed, in accordance with various embodiments. The algorithm receives, as input, a current Q function estimate. It has various methods available to it, such as “Sample,” which draws a sample of transitions (i.e. state/action/state triples, as described before, with an associated reward) from, e.g., local memory 114, “Fit,” which fits a random forest model to minimize root mean square error on a training set, and “Acts,” which obtains allowable task/step actions that may be selected based on a current state of subject 102. The algorithm also includes a number of parameters, including K (number of learning iterations, may be greater than or equal to 1), γ (future reward discount factor), and N (experience sample size). The algorithm may output a new Q function estimate determined, for instance, using the operations shown in FIG. 2 under “Method:”. With a sufficiently large sample of transitions, the algorithm will, in probability, converge to an estimate of the optimal Q function; i.e. the expected cumulative reward from taking an action in a state, then following the best possible policy afterwards.

In some embodiments, all prompts (or more generally, actions) provided to subject 102 may be assigned an intrusiveness score, I, e.g., from zero (least intrusive) to one (most intrusive). A reward of (1−I)×R, where R is a positive constant, may be received/generated when subject 102 provides input that is responsive to a prompt, e.g., to trigger a task, advance a step through a task (e.g., i.e. task-engagement input), completing the task, etc. A penalty P (which can be a negative constant) may be received/generated when subject 102 rejects a prompt (or more generally, an action), which may occur, for instance, when subject 102 does not trigger an offered task (e.g., enters the kitchen and is offered instructions to cook a meal, but declines), or aborts a task midstream. Other rewards and/or penalties may be associated with attributes of the subject's responsive input, such as time required for subject 102 to complete a step of a task.

In other embodiments, learning engine 112 may employ other learning techniques. For example, in some embodiments, policy 116 may include one or more artificial neural networks that are trained to select an action based on a state of subject 102. For example, various features of a state of subject 102 may be applied as input across the neural network to generate output. In some embodiments, the output may include probabilities associated with a plurality of potential actions (e.g., prompts to be provided to subject 102) that may be taken, e.g., by controller 108, in response to the subject state. In some embodiments, the highest probability action may simply be selected. In other embodiments, an action may be stochastically selected based on the probabilities, meaning the highest probability action is the most likely to be selected, but another action could be randomly selected instead.

In various embodiments, such a neural network may be trained using reinforcement learning, e.g., to adjust one or more weights associated with hidden layer(s) of the neural network, and ultimately, the output probabilities associated with the potential actions. For example, a cumulative reward may be computed based on a session between subject 102 and master device 104 (or slave device 106) that leads to some outcome for a particular task. If the outcome of the task is failure, the reward value may be minimal or negative. If the outcome of the task is success, the reward value may be positive. In some embodiments, the number of “turns” required to achieve a positive outcome may be considered, e.g., as a penalty that may affect the cumulative reward value.

Additionally or alternatively, the number of turns required may affect a reward that is associated with each state/action/state triple processed during the task, e.g., for training purposes. For example, suppose it takes ten turns for subject 102 to successfully complete a task. At each turn, a state of subject 102 was applied as input across the neural network to generate output that was used to select the next action; hence, each turn is associated with a state/action/state triple. The cumulative reward calculated at completion of the task may be applied most heavily at later turns, e.g., because those later turns may have played a relatively large role in successful completion of the task by subject 102. In contrast, the cumulative reward may be reduced further upstream as it is applied to earlier state/action/state triples because those state/action/state triples likely played smaller roles in the ultimate outcome. And as noted above, in some embodiments, an intrusiveness score associated with each action of a state/action/state triple may be taken into account, for instance, when applying a cumulative reward value to that state/action/state triple.

Outside of influencing/adapting policy 116, a measure of impairment computed for subject 102 may be used for other purposes. If a subject's estimated level of impairment changes, particularly if it appears the subject is deteriorating, caregivers and/or clinicians may be notified, e.g., via audio/visual output, and/or by email, text message, or other push notifications. For example, in some embodiments, times required for subject 102 to provide task-engagement input in response to prompts (e.g., indicating completion of a step of a task) and/or statistics computed based on those times may be evaluated to determine a measure of impairment of subject 102. In some embodiments, one or more of these times and/or statistics may be applied as input across various types of machine learning classifiers, such as artificial neural networks or support vector machines, to classify the subject as having a particular level of impairment. In some embodiments, such a machine learning classifier such as an artificial neural network may be trained with training examples that include response times and/or associated statistics for subjects with known levels of impairment. The known levels of impairment may be used as labels for the training examples. The training examples may be applied across an untrained neural network to generate output, which is then compared with the labels indicating the subjects' known levels of impairment. Any difference between the labels and the output may be determined and used to train the neural network, e.g., using techniques such as back propagation and/or stochastic/batch gradient descent.

In another aspect, it may be beneficial to ensure that subjects, particularly those suffering from relatively severe impairment, continue to receive guidance for performing IADL tasks even if technological issues arise. That is why, in some embodiments, computing devices available to subject 102 may be organized as master devices (104) and slave devices (106). Referring once again to FIG. 1, in various embodiments, master device 104 and one or more slave device(s) 106 may be in network communication, e.g., using Wi-Fi or other similar communication technologies. In various embodiments, slave device 106 may be configured to perform much of the same functionality as master device 104. For example, slave device 106 may also include a user interaction engine 110, a controller 108, and local memory 114, each which may serve a function similar to that described previously with respect to master device 104. In some embodiments, whichever device, be it master device 104 or slave device 106, that subject 102 engages with during a particular session may communicate with the other devices to ensure all devices are able to continue to provide an amount and/or types of guidance, and that the data (e.g., policy 116) used by all the devices remains consistent.

However, communication networks such as Wi-Fi can fail for a variety of reasons, such as power outage, hardware failure, etc. Moreover, individual computing devices may fail, e.g., because they run out of power, experience hardware failure, are dropped, etc. In such scenarios, subject 102 may still need to be able to perform IADLs with guidance provided using techniques described herein. Accordingly, in various embodiments, should a communication network between the various devices fail, and/or should one or more devices themselves fail (especially master device 104), slave device(s) 106 may be configured to continue operating autonomously, using their own copies of local memory 114 and/or policy 116, to provide guidance to subject 102 for performing IADL tasks, and to monitor response times by subject 102. Consequently, when network communication is reestablished, master device 104 and slave device(s) 106 may once again synchronize their data (e.g., state/action/state triples in local memory 114 and policy 116) so that they operate consistently, and so that global memory 118 may be updated to include the most recent versions of policy 116 and state/action/state triples.

FIGS. 3A-D depict examples of visual output that may be provided (as an “action” in response to a subject's state) to a subject upon the subject being detected entering a kitchen in the morning. In FIG. 3A, a graphical user interface includes two prompts, 332A and 332B, that provide options of tasks the subject may perform: “Make Oatmeal” and “Take Medication,” respectively. Prompts 332A and 332B may be output, e.g., by a (master or slave) computing device in the kitchen or carried by the subject (e.g., a smart phone, tablet, smart glasses, etc.), upon detection by one or more presence sensors 120 of the subject in the kitchen in the morning. In particular, the subject's state may include a location of “kitchen” and a current time that corresponds to times in which the subject typically makes breakfast and/or takes medication(s). Based on policy 116, controller 108 may determine, based on the subject's state, that prompts 332A and 332B should be presented. While visual output is depicted in FIGS. 3A-D, this is not meant to be limiting. In various embodiments, output may additionally or alternatively include audio output and/or haptic output.

Suppose the subject selects prompt 332A to “Make Oatmeal.” This may trigger a “making oatmeal” task that includes a number of steps required to make oatmeal. Two such steps are represented by prompts 332C and 332D in FIG. 3B, “Get Oatmeal” and “Locate Pan.” In some embodiments in which actions are customized to the subject's home, these visual prompts may further indicate (e.g., with a picture) a location of these items, such as in an actual cupboard of the subject's kitchen. Two prompts 332C and 332D are depicted simultaneously in FIG. 3B because these steps of the “making oatmeal” tasks are not order specific. In addition, the prompts 332C and 332D are fairly specific, and may be selected for presentation to a subject having relatively severe cognitive impairment. A less-impaired subject may not be shown one or more of prompts 332C and/or 332D because the less-impaired subject may be expected to be able to perform these steps without guidance.

Once the subject provides task-engagement input that indicates completion of the steps represented by prompts 332C and 332D, e.g., by tapping the prompts 332C/332D on a touchscreen, the subject may be presented with prompt 332E shown in FIG. 3C that instructs the subject how to cook the oatmeal (“Cook for 1 min on high, while stirring”). In some embodiments, a timer may be set automatically, e.g., in association with prompt 332E, that informs the subject when the allotted cook time has elapsed. The subject may indicate completion of the step represented by prompt 332E, e.g., by tapping prompt 332E on a touchscreen or by voice control. This may cause prompt 332F in FIG. 3D to be presented. Prompt 332F reminds the subject to turn off the stove, perhaps the most important step of the “making oatmeal” task for a cognitively-impaired subject. If the subject fails to provide task-engagement input for prompt 332F, e.g., after some predetermined time interval, an alarm may be raised, e.g., to the subject and/or to one or more caregivers and/or clinicians, that the stove may still be operating. While not depicted in FIGS. 3A-D, other types of prompts may be output to the subject, such as prompts offering congratulations for completing IADL tasks/steps.

As discussed previously, in various embodiments, interactions between the subject and the various prompts 332 (i.e., between subject 102 and user interaction engine 110) may be recorded and evaluated to determine, on an ongoing basis, a measure of the subject's impairment. These data may be recorded in local database 114 and/or in global database 118. When slave device(s) 106 are present, these data may be pushed to their respective local database(s) 114 as well.

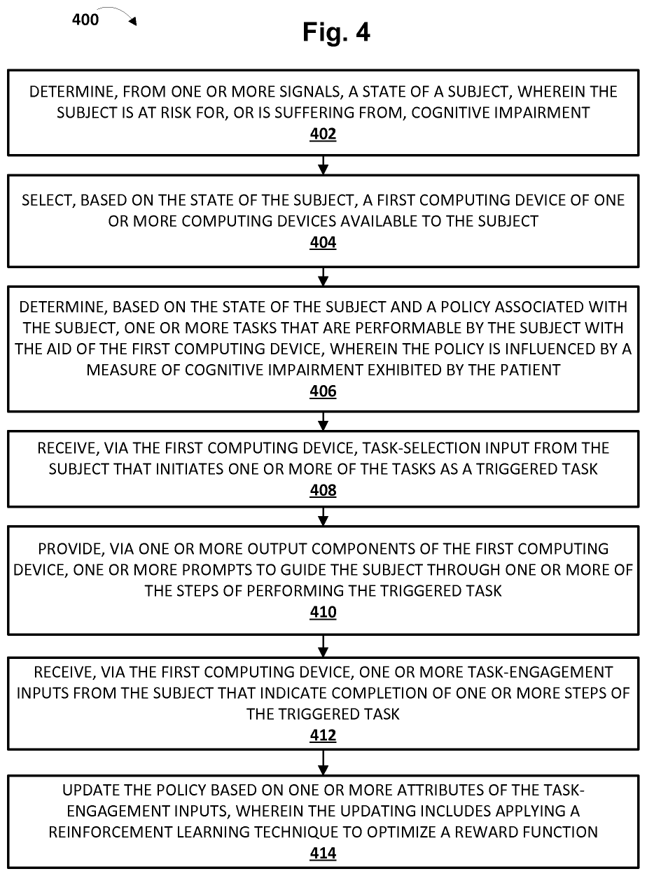

FIG. 4 depicts an example method 400 for practicing selected aspects of the present disclosure, in accordance with various embodiments. For convenience, the operations of the flow chart are described with reference to a system that performs the operations. This system may include various components of various computer systems, including master device 104 and/or slave device 106. Moreover, while operations of method 400 are shown in a particular order, this is not meant to be limiting. One or more operations may be reordered, omitted or added.

At block 402, the system may determine, e.g., from one or more signals (e.g., from presence sensor 120, current time, current date, etc.), a state of a subject that is at risk for, or is suffering from, impairment such as cognitive impairment. At block 404, the system may select, based on the state of the subject, a first computing device of one or more computing devices available to the subject. This may include, for instance, the nearest computing device to the subject's last-known location and/or a computing device carried by the subject.

At block 406, the system may determine, e.g., based on the state of the subject and a policy (e.g., 116) associated with the subject, one or more tasks that are performable by the subject with the aid of the first computing device. As noted above, in various embodiments, the policy is influenced by a measure of cognitive impairment exhibited by (e.g., observed in) the subject. These may be presented to the subject, e.g., as depicted in FIG. 3A. At block 408, the system may receive, via the first computing device or via another computing device, task-selection input from the subject that initiates one or more of the tasks as a triggered task, e.g., by selecting one of prompts 332A/332B in FIG. 3A.

At block 410, the system may, as actions that are responsive to the subject's state determined at block 402, provide, e.g., via one or more output components of the first computing device, one or more prompts to guide the subject through one or more of the steps of performing the triggered task. Examples of such prompts are depicted in FIGS. 3B-D. At block 412, the system may receive, via the first computing device or via another computing device, one or more task-engagement inputs from the subject that indicate completion of one or more steps of the triggered task. This task-engagement input may include, for instance, the subject tapping a visual prompt, swiping a visual prompt, providing some gesture that may be detected by a camera, natural language input from the subject (“OK, I've located the pot,” “OK, I've turned off the stove”), etc.

At block 414, the system, e.g., by way of learning engine 112, may update the policy based on one or more attributes of the task-engagement inputs. These attributes may include response times, response time statistics, outcomes, etc. As described previously, in some embodiments, updating the policy may include applying a reinforcement learning technique to optimize a reward function. Also as described previously, the policy may be updated based on other signals as well, such as attributes of the prompts provided at block 410. For example, intrusiveness measures associated with the prompts may be used, e.g., as penalties, to alter a reward value that is ultimately used for reinforcement learning that updates the policy.

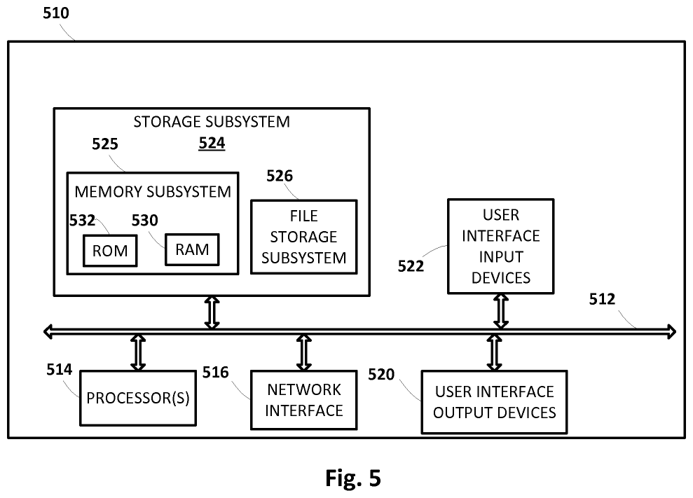

FIG. 5 is a block diagram of an example computer system 510. Computer system 510 typically includes at least one processor 514 which communicates with a number of peripheral devices via bus subsystem 512. These peripheral devices may include a storage subsystem 524, including, for example, a memory subsystem 525 and a file storage subsystem 526, user interface output devices 520, user interface input devices 522, and a network interface subsystem 516. The input and output devices allow user interaction with computer system 510. Network interface subsystem 516 provides an interface to outside networks and is coupled to corresponding interface devices in other computer systems.

User interface input devices 522 may include a keyboard, pointing devices such as a mouse, trackball, touchpad, or graphics tablet, a scanner, a touchscreen incorporated into the display, audio input devices such as voice recognition systems, microphones, and/or other types of input devices. In general, use of the term “input device” is intended to include all possible types of devices and ways to input information into computer system 510 or onto a communication network.

User interface output devices 520 may include a display subsystem, a printer, a fax machine, or non-visual displays such as audio output devices. The display subsystem may include a cathode ray tube (CRT), a flat-panel device such as a liquid crystal display (LCD), a projection device, or some other mechanism for creating a visible image. The display subsystem may also provide non-visual display such as via audio output devices. In general, use of the term “output device” is intended to include all possible types of devices and ways to output information from computer system 510 to the subject or to another machine or computer system.

Storage subsystem 524 stores programming and data constructs that provide the functionality of some or all of the modules described herein. For example, the storage subsystem 524 may include the logic to perform selected aspects of method 400, and/or to implement one or more components depicted in the various figures. Memory 525 used in the storage subsystem 524 can include a number of memories including a main random access memory (RAM) 530 for storage of instructions and data during program execution and a read only memory (ROM) 532 in which fixed instructions are stored. A file storage subsystem 526 can provide persistent storage for program and data files, and may include a hard disk drive, a CD-ROM drive, an optical drive, or removable media cartridges. Modules implementing the functionality of certain implementations may be stored by file storage subsystem 526 in the storage subsystem 524, or in other machines accessible by the processor(s) 514.

Bus subsystem 512 provides a mechanism for letting the various components and subsystems of computer system 510 communicate with each other as intended. Although bus subsystem 512 is shown schematically as a single bus, alternative implementations of the bus subsystem may use multiple busses.

Computer system 510 can be of varying types including a workstation, server, computing cluster, blade server, server farm, smart phone, smart watch, smart glasses, set top box, tablet computer, laptop, or any other data processing system or computing device. Due to the ever-changing nature of computers and networks, the description of computer system 510 depicted in FIG. 5 is intended only as a specific example for purposes of illustrating some implementations. Many other configurations of computer system 510 are possible having more or fewer components than the computer system depicted in FIG. 5.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms. The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited. In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method implemented by one or more processors, comprising:
determining, from one or more signals, a state of a subject, wherein the subject is at risk for, or is suffering from, cognitive impairment;
selecting, based on the state of the subject, a first computing device of one or more computing devices available to the subject;
determining, based on the state of the subject and a policy associated with the subject, one or more tasks that are performable by the subject with the aid of the first computing device, wherein the policy is influenced by a measure of cognitive impairment exhibited by the subject;
receiving, via the first computing device, task-selection input from the subject that initiates one or more of the tasks as a triggered task;
receiving, via the first computing device, one or more task-engagement inputs from the subject that indicate completion of one or more steps of the triggered task; and
updating the policy based on one or more attributes of the task-engagement inputs, wherein the updating includes applying a reinforcement learning technique to optimize a reward function.

2. The method of claim 1, further comprising providing, via one or more output components of the first computing device, one or more prompts to guide the subject through one or more of the steps of performing the triggered task, wherein the one or more prompts are selected based at least in part on the policy associated with the subject, wherein updating the policy further includes updating the policy based at least in part on one or more attributes of the one or more prompts.

3. The method of claim 2, wherein the one or more attributes of the one or more prompts include a measure of intrusiveness.

4. The method of claim 1, wherein the one or more signals includes a signal from a presence sensor, and the state includes at least a last-detected location of the subject determined based on the signal from the presence sensor.

5. The method of claim 1, wherein the first computing device is further selected based on the policy associated with the subject.

6. The method of claim 1, wherein the reinforcement learning technique comprises a random forest batch-fitted Q learning algorithm.

7. The method of claim 1, wherein the reinforcement learning technique comprises an artificial neural network.

8. The method of claim 1, wherein the one or more attributes of the task-engagement inputs include a reward or penalty determined based on a response time by the subject to provide a given task-engagement input of the task-engagement inputs.

9. The method of claim 1, wherein the triggered task includes preparation of a meal.

10. The method of claim 1, wherein the triggered task includes one or more of oral hygiene maintenance, medication ingestion, and adorning of clothing.

11. A system comprising one or more processors and memory operably coupled with the one or more processors, wherein the memory stores instructions that, in response to execution of the instructions by one or more processors, cause the one or more processors to perform the following operations:
determining, from one or more signals, a state of a subject, wherein the subject is at risk for, or is suffering from, cognitive impairment;
selecting, based on the state of the subject, a first computing device of one or more computing devices available to the subject;
determining, based on the state of the subject and a policy associated with the subject, one or more tasks that are performable by the subject with the aid of the first computing device, wherein the policy is influenced by a measure of cognitive impairment exhibited by the subject;
receiving, via the first computing device, task-selection input from the subject that initiates one or more of the tasks as a triggered task;
receiving, via the first computing device, one or more task-engagement inputs from the subject that indicate completion of one or more steps of the triggered task; and
updating (414) the policy based on one or more attributes of the task-engagement inputs, wherein the updating includes applying a reinforcement learning technique to optimize a reward function.

12. The system of claim 11, further comprising instructions for providing, via one or more output components of the first computing device, one or more prompts to guide the subject through one or more of the steps of performing the triggered task, wherein the one or more prompts are selected based at least in part on the policy associated with the subject, wherein updating the policy further includes updating the policy based at least in part on one or more attributes of the one or more prompts.

13. The system of claim 11, wherein the one or more attributes of the one or more prompts include a measure of intrusiveness.

14. The system of claim 11, wherein the one or more signals includes a signal from a presence sensor, and the state includes at least a last-detected location of the subject determined based on the signal from the presence sensor.

15. The system of claim 11, wherein the first computing device is further selected based on the policy associated with the subject.

16. The system of claim 11, wherein the reinforcement learning technique comprises a random forest batch-fitted Q learning algorithm.

17. The system of claim 11, wherein the reinforcement learning technique comprises an artificial neural network.

18. The system of claim 11, wherein the one or more attributes of the task-engagement inputs include a reward or penalty determined based on a response time by the subject to provide a given task-engagement input of the task-engagement inputs.

19. The system of claim 11, wherein the triggered task includes preparation of a meal.

20. At least one non-transitory computer-readable medium comprising instructions that, in response to execution of the instructions by one or more processors, cause the one or more processors to perform the following operations:
determining, from one or more signals, a state of a subject, wherein the subject is at risk for, or is suffering from, cognitive impairment;
selecting, based on the state of the subject, a first computing device of one or more computing devices available to the subject;
determining, based on the state of the subject and a policy associated with the subject, one or more tasks that are performable by the subject with the aid of the first computing device, wherein the policy is influenced by a measure of cognitive impairment exhibited by the subject;
receiving, via the first computing device, task-selection input from the subject that initiates one or more of the tasks as a triggered task;
receiving, via the first computing device, one or more task-engagement inputs from the subject that indicate completion of one or more steps of the triggered task; and
updating the policy based on one or more attributes of the task-engagement inputs, wherein the updating includes applying a reinforcement learning technique to optimize a reward function.
